(19) United States
(12) Patent Application Publication
(10) Pub. No.: US 2021/0253732 A1
(43) Pub. Date: Aug. 19, 2021

(54) ANTIGEN-BINDING FRAGMENT AND/OR APTAMER FOR BINDING TO AN EXTRACELLULAR PART OF CD9 AND THERAPEUTIC USES

(71) Applicants: Aurelio LORICO, Las Vegas, NV (US); Denis CORBEIL, Dresden (DE); Mark F SANTOS, Las Vegas, NV (US); Germana A RAPP, Las Vegas, NV (US)

(72) Inventors: Aurelio LORICO, Las Vegas, NV (US); Denis CORBEIL, Dresden (DE); Mark F SANTOS, Las Vegas, NV (US); Germana A RAPP, Las Vegas, NV (US)

(21) Appl. No.: 17/271,690

(22) PCT Filed: Aug. 29, 2019
(86) PCT No.: PCT/IB2019/057294

§ 371 Date: Feb. 26, 2021

Related U.S. Application Data
(60) Provisional application No. 62/724,183, filed on Aug. 29, 2018.

(30) Foreign Application Priority Data
Aug. 29, 2018 (GB) 1814065.7

(51) Int. Cl. C07K 16/28; C12N 15/115; A61K 45/06

(52) U.S. Cl. CPC C07K 16/2896; C12N 15/115; C12N 2310/16; C07K 2317/55; C07K 2317/76; A61K 45/06

(57) ABSTRACT

Disclosed is an antigen-binding fragment (Fab) and/or an aptamer that recognises a target epitope in an extracellular part of CD9, wherein the Fab and/or the aptamer bind directly to the extracellular part of the CD9, and wherein the Fab binds to the extracellular part of the CD9 at a similar location as that of the aptamer. Specifically, the Fab is a fragment of a monoclonal antibody and has a size of approximately 40-60 kDa, 45-55 kDa, 48-52 kDa or 50 kDa, and wherein the monoclonal antibody is at least one of an anti-human CD9 and an anti-CD9. Also, disclosed is a biological molecular assay that recognises an epitope in an extracellular part of a CD9. The biological molecular assay includes use of an antigen-binding fragment (Fab) and an aptamer.

### TECHNICAL FIELD

The present disclosure relates generally to inhibiting intercellular communications for preventing or treating cancer and infectious diseases.

Specifically, the present disclosure relates to antigen-binding fragments (Fab) and/or aptamers that recognise target epitopes in extracellular part of CD9 and their use in therapy. The present disclosure also relates to pharmaceutical compositions comprising an antigen-binding fragments (Fab) and/or aptamers, and at least one pharmaceutically acceptable excipient, adjuvant or carrier. Furthermore, the present disclosure relates to biological molecular assays that recognise target epitopes in the extracellular part of CD9 by using the aforesaid antigen-binding fragments (Fab) and/or aptamers.

### BACKGROUND

Every year, millions of deaths are caused by cancer worldwide. As part of the multiple approaches to cancer treatment, the role of extracellular vesicles in microenvironmental transformation of tumours has been evaluated. Extracellular membrane vesicles, or EVs, mediate intercellular communication between a recipient (namely, a host) cell and a donor cell. EVs are endocytosed by a host cell and subsequently the endocytosed EV-associated proteins are transported into the cytoplasm and nucleoplasm of the host cell.

Conventionally, various targeted drugs and immunotherapeutic strategies have been explored for treating different types of cancers. Among different approaches that have been conventionally adopted for curing cancer, there are approaches such as identification of various cellular components as targets for delivery of therapeutic agents to cancer cells and inhibiting cell multiplication process, treatments based on chemotherapy as well as antiangiogenic agents.

However, such treatments based on conventional pharmacological or immunotherapeutic strategies have transient responses and, on account of a highly adaptable nature of tumours, may result in development of drug (and/or radiation)-resistance over time and as a consequence, a decline in effectiveness of such treatments in curing cancer. Such strategies may even make the tumours more aggressive, leading to their progression to other parts of the body or advanced stages of the disease.

Therefore, there exists a need to find new approaches for the effective treatment of cancer and other conditions.

### SUMMARY

The present disclosure seeks to provide an antigen-binding fragment (Fab) and/or an aptamer that recognizes a target epitope in an extracellular part of CD9. The present disclosure also seeks to provide a pharmaceutical composition comprising an antigen-binding fragment (Fab) and/or an aptamer as defined herein, and at least one pharmaceutically acceptable excipient, adjuvant or carrier.

Moreover, the present disclosure seeks to provide a biological molecular assay that recognises the target epitope in an extracellular part of CD9.

In a first aspect, embodiments of the present disclosure relate to an antigen-binding fragment (Fab) and/or an aptamer that recognises a target epitope in an extracellular part of CD9, wherein the Fab and the aptamer bind directly to the extracellular part of the CD9, and wherein the Fab binds to the extracellular part of the CD9 at a similar location as that of the aptamer.

An advantage of the present invention is the provision of directly targeting the extracellular membrane vesicles (EVs) implicated in mediating the intercellular communications between donors (cancer cells, infected cells or infectious agents) and recipient (host) cells.

Optionally, the Fab is a fragment of a monoclonal antibody and has a size of approximately 40-60 kDa, 45-55 kDa, 48-52 kDa or 50 kDa, and wherein the monoclonal antibody is at least one of an anti-human CD9 and an anti-CD9.

Optionally, the monoclonal antibody is an antibody derived from at least one of antigens including TSPAN29, DRAP-27, BTCC-1, motility related protein-1, leukocyte antigen MIC3, 5H9, MRP-1 and BA-2/P24.

More optionally, the fragment of the monoclonal antibody (CD9 Fab) is derived from a 5H9 anti-CD9 monoclonal antibody, optionally wherein the fragment is derived by digestion of the antibody with papain.

Optionally, the 5H9 anti-CD9 monoclonal antibody fragment binds directly to the extracellular part of the CD9.

Optionally, the aptamer is made of DNA or RNA and the aptamer is a CD9 aptamer.

Optionally, the antigen-binding fragment (Fab) and/or an aptamer inhibits an intercellular communication in multicellular organisms by interfering with at least one of:
(i) an endocytosis of extracellular membrane vesicles (EVs);
(ii) a fusion of EVs with cells; and
(iii) an intracellular transport of endocytosed biomaterials in recipient cells.

Optionally, the antigen-binding fragment (Fab) and/or an aptamer is for use in at least one of: medicine, veterinary medicine, agriculture or as an insecticide.

More optionally, the antigen-binding fragment (Fab) and/or an aptamer is for use as a medicament.

Optionally, the antigen-binding fragment (Fab) and/or an aptamer is for use in a treatment and/or prevention of a disease or condition in which the endocytosis of extracellular vesicles is implicated, optionally, wherein a subject is a human.

Optionally, the disease includes at least one of cancer, cancer metastasis and an infection.

Optionally, the infection is caused by a virus. More optionally, the virus is an enveloped virus. Furthermore, optionally, the virus is HIV-1.

Optionally, the disease is a neurodegenerative disease.

Optionally, the disease is Alzheimer's disease, Parkinson's disease, ventricular hypertrophy, a type I diabetes, a type II diabetes, a kidney disease, macular degeneration or a lung disease.

In another aspect, embodiments of the present disclosure provide a pharmaceutical composition comprising an antigen-binding fragment (Fab) and/or an aptamer, and at least one pharmaceutically acceptable excipient, adjuvant or carrier.

The aforesaid antigen-binding fragment (Fab) and/or aptamer and the aforementioned pharmaceutical composition, as disclosed in the present disclosure, provide an efficacious way to inhibit intercellular communications by inactivating the extracellular membrane vesicles (EVs) outside a host cell. Typically, the antigen-binding fragment (Fab) and/or aptamer recognise the target epitopes in the extracellular part of the CD9, expressed by almost all extracellular membrane vesicles (EVs). Furthermore, the Fab and/or the aptamer bind directly to the recognised CD9. Notable, the CD9 mediates the uptake of EVs by the host cell, and is therefore a potential target for blocking the intracellular communication and thus the uptake of EVs by the host cells. This way the CD9 is devoid of its property of assisting the endocytosis of EVs and nuclear transfer of their biocomponents in a host cell of a subject organism. Beneficially, the present disclosure provides a therapeutic approach to target the intercellular communications. Typically, disruption of such intercellular communications may be a powerful and fruitful strategy to combat the potential diseases such as cancer, cancer metastasis and infections. Furthermore, since such intercellular communications are implicated in a plurality of other diseases, their disruptions thereby enable effective therapy of a plurality of other diseases, such as neurodegenerative disease (such as Alzheimer's disease, Parkinson's disease and the like), diabetes (type I and type II), ventricular hypertrophy, lung diseases, and so forth.

Furthermore, the aforementioned pharmaceutical composition is for use in combination with one or more conventional treatment strategies, including therapeutic agents, to prevent a plurality of diseases. There is thereby provided a novel approach that involves the disruption of the intercellular communication implicated in endocytosis of EVs to cause fatal diseases. Moreover, this novel approach helps to overcome the problem of treating cancer and other infectious and age-related diseases by targeting the delivery of the EVs to recipient (namely, host) cells. Furthermore, the ability to specifically target the EVs on the donor cells as well on the surface of host cells makes the entire treatment more cost-effective, less painful and also slows down the development of advanced stages of the disease. A lot of human suffering is thereby avoided, to great benefit to human society.

Optionally, the pharmaceutical composition is for use in the treatment and/or prevention of the disease or condition selected from cancer, cancer metastasis, infection, the neurodegenerative disease, Alzheimer's disease, Parkinson's disease, ventricular hypertrophy, the type I diabetes, the type II disease, the kidney disease, macular degeneration and the lung disease.

Optionally, the antigen-binding fragment (Fab), the aptamer or the pharmaceutical composition is for use when the patient is further administered one or more therapeutic agents or when the Fab and/or the aptamer is provided in combination with one or more therapeutic agents.

Optionally, the pharmaceutical composition is for use in combination with one or more therapeutic agents for the treatment and/or prevention of the disease or condition.

In yet another aspect, embodiments of the present disclosure provide a biological molecular assay that recognises a target epitope in an extracellular part of CD9, wherein the biological molecular assay includes use of an antigen-binding fragment (Fab) and/or an aptamer, and wherein the Fab and the aptamer bind directly to the extracellular part of the CD9, and wherein the Fab binds to the extracellular part of the CD9 at a similar location as that of the aptamer.

Optionally, the Fab is a fragment of a monoclonal antibody and has a size of approximately 40-60 kDa, 45-55 kDa, 48-52 kDa or 50 kDa, and wherein the monoclonal antibody is at least one of an anti-human CD9 and an anti-CD9.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

A better understanding of the present invention may be obtained through the following examples which are set forth to illustrate but are not to be construed as limiting the present invention.

### BRIEF DESCRIPTION OF DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIGS. 1A and 1B are pictorial representations of 5H9 anti-CD9 monoclonal antibody (mAb) or CD9 antigen-binding fragment (CD9 Fab) and the corresponding antigen, i.e. transmembrane protein CD9 (A), and the generation of CD9 antigen-binding fragment (CD9 Fab) by papain digestion. Protein A incubation depletes fragment crystalline (Fc);

FIG. 1C is a schematic illustration of a micrograph image depicting full-size 5H9 anti-CD9 monoclonal antibody (Ab, before) and Fab fragments thereof upon papain digestion (after);

FIGS. 1D and 1E are graphical representation of data depicting the binding of 5H9 anti-CD9 monoclonal antibody (CD9 Fab) to melanoma cells in presence and absence of CD9 antigen-binding fragment (CD9 Fab) at an antibody concentration of 25 μg/ml (D) and 50 μg/ml (E);

FIGS. 2A, 2B and 2C are graphical representation of data depicting significance of CD9 antigen-binding fragment (CD9 Fab) in the uptake of extracellular membrane vesicles (EVs) by melanoma (FEMX-I) cells;

FIGS. 2D and 2E are graphical representation of data depicting significance of CD9 antigen-binding fragment (CD9 Fab) in the nuclear transfer of EV-derived CD9-GFP by melanoma (FEMX-I, D) cells and mesenchymal stem cells (MSC, E) respectively;

FIGS. 3A to 3C and 3D to 3F are graphical representation of data depicting significance of CD9 antigen-binding fragment (CD9 Fab) in the uptake of EVs by melanoma cells, A375 (A-C) and C8161 (D-F);

FIGS. 3G and 3H are graphical representation of data depicting significance of CD9 antigen-binding fragment (CD9 Fab) in the nuclear transfer of EV-derived CD9-GFP by melanoma cells, A375 (G) and C8161 (H);

FIGS. 4A and 4B are graphical representation of data depicting dose-dependent inhibition of CD9 antigen-binding fragment (CD9 Fab) on the uptake of EVs (A) and nuclear transfer (B) of EV-derived CD9-GFP by melanoma (FEMX-I) cells;

FIGS. 5A and 5B are pictorial representations displaying a positive (A) and negative (B) impact of 5H9 anti-CD9 monoclonal antibody (CD9 Ab) and CD9 antigen-binding fragment (CD9 Fab) and/or aptamer, respectively, on the endocytosis of CD9-containing EVs;

FIG. 6A is pictorial representation of immunoblotting depicting quantification of CD9 and β-actin in melanoma cell lines (FEMX-I, A375 and C8161) and primary mesenchymal stem cells (MSC);

FIG. 6B is a graphical representation of data depicting quantification of CD9 and β-actin in melanoma (FEMX-I, A375 and C8161) cells and primary mesenchymal stem cells (MSC); and

FIG. 6C is a graphical representation of data of a micrograph depicting a number of cell surface CD9 per cell in melanoma cell lines (FEMX-I, A375 and C8161) and primary mesenchymal stem cells (MSC).

FIG. 7 is a graphical representation of data displaying the impact of CD9 antigen-binding fragment (CD9 Fab) on the infection of HIV virus into recipient cells.

LIST OF ABBREVIATIONS

| Abbreviation | Meaning |
|---|---|
| CD9 Fab | Fragment of monoclonal antibody against CD9 |
| CLSM | Confocal laser scanning microscopy |
| EVs | Extracellular membrane vesicles |
| Fab | Antigen-binding fragment |
| mAb | Monoclonal antibody |
| MSC | Mesenchymal stromal cell |

### Definitions

As used herein, the following terms shall have the following meaning:

As used herein, the term “antibody” as used herein refers generally to a molecule that contains at least one antigen-binding site. The antibody includes, but is not limited to a native antibody, a fragment of the native antibody, a peptibody, an antibody mimetic and variants thereof. Furthermore, the binding of an antibody to its specific antigen may cause a variety of effects, including, but not limited to, modulation, reduction, enhancement, antagonizing, agonizing, mitigation, alleviation, blocking, inhibition, abrogation and interference with binding in vitro, in situ and/or in vivo.

As used herein, the term “antigen” refers to a molecule, generally a toxin or other foreign substance, capable of inducing an immune response in a subject organism (namely, a host), such as a human or an animal. Sometimes, antigens are a part of the host organism itself, responsible to induce an autoimmune response (namely, a disease or condition) in the host organism.

Furthermore, as used herein, the term “antigen” refers to a molecular target of an antibody, which is expressed in a tissue, a cell or an extracellular membrane vesicle (EV) or secreted in body fluids such as urine, saliva, tear, blood, seminal and cerebrospinal fluids.

As used herein, the term “antigen-binding fragment (Fab)” as used herein refers to a fragment of an antibody that binds to an antigen.

As used herein, the term “extracellular membrane vesicles (EVs)” refers to small vesicles released from almost all types of multicellular organisms. EVs are exosomes, exosome-like, ectosomes, microvesicles, apoptotic bodies, midbody or any other membrane particles released by, or budding off from, cells. Specifically, the EVs serve as effective means for intercellular communication. Generally, EVs transfer specific bioactive molecules, comprising functional nucleic acids including mRNAs and microRNAs (miRNAs) across cells for their translation into homologous or heterologous proteins. Proteins and lipids are also bioactive molecules associated with EVs. Moreover, the EVs are recognised as antigens carriers and also as potential immunotherapeutic targets. Typically, over 98% of EVs express cell surface antigens or cell surface determinants or cluster of differentiation (CD). Specifically, the EVs are involved in the intercellular signalling, production of proteins and removal of unwanted proteins and transfer of pathogens across a plurality of cells.

As used herein, the term “CD9” refers to a glycoprotein antigen, belonging to a tetraspanin family of proteins. The CD9 is composed of four hydrophobic transmembrane domains (each in a range of 22 to 27 amino acids), a N-terminal cytoplasmic tail (range of 5 to 20 amino acids), a C-terminal cytoplasmic tail (range of 2 to 12 amino acids), a short intracellular part (range of 3 to 8 amino acids), and two extracellular parts (namely, domain or loop). The first loop contains about 20 residues (range from 10 to 30) while the second one about 83 (range from 70 to 90). An alternative splice variant with five transmembrane domains has been identified. Furthermore, the CD9 contains a tetraspanin signature (in a range of 65 to 89 amino acids) and a CCG motif (in a range of 152 to 154 amino acids). Typically, the CD9 is expressed by a variety of hematopoietic cells (platelets, eosinophils, neutrophils, basophils, monocytes and erythrocytes), epithelial cells, spinal motoneurons, foetal (fetal) neurons and activated B and T lymphocytes. CD9 is expressed by cancer cells. CD9 is associated with EVs. The CD9 forms multimeric complexes with other cell surface proteins, such as other tetraspanins, integrins, pregnancy related glycoproteins, transforming growth factor alpha (TGF-α), MHC Class II molecules, Ig superfamily, and the like.

Consequently, the CD9 undergoes a series of chemical modifications, including but not limited to, glycosylation, acylation and phosphorylation. Furthermore, the CD9 is involved in cell adhesion, cell proliferation, cell motility, T-cell activation, osteoclastogenesis, signal transduction, formation of paranodal junctions, gamete fusion, muscle cell fusion, maintenance of myotubes, and so forth.

As used herein, the term “extracellular part of CD9” or “extracellular part of the CD9” refers to a part of the CD9, projecting outwards as loops. The extracellular part of the CD9 includes a smaller extracellular part (range from 10 to 30 amino acids) and a larger extracellular part (range from 70 to 90 amino acids).

As used herein, the term “aptamer” as used herein refers to an oligonucleotide molecule or a short sequence of peptide, which bind to a specific target molecule, such as the CDs. The aptamers enable substantial recognition of the molecular targets when compared to the corresponding antibodies, and therefore binds to the molecular targets with high affinity and specificity. The aptamers are readily produced by chemical synthesis and engineered based on the properties of the molecular target. Furthermore, the aptamers can be selected using molecular evolution techniques, such as systematic evolution of ligands by exponential enrichment (SELEX) or in vitro selection from different molecular libraries.

As used herein, the term “biological molecular assay” refers to an immunoassay. Typically, the biological molecular assays may comprehend one or more of: receptor binding; protein interactions with proteins, carbohydrates, lipids and nucleic acids; gene expression; and enzymatic catalysis. The biological molecular assays are also able to identify EVs and other biomolecules implicated in causing a disease. Generally, such immunoassays exploit principles of luminescence, fluorescence, colorimetric and the like to investigate biomolecules in an analyte.

As used herein, the term “analyte” refers to a substance that is being analysed by implementing the biological molecular assay. The analyte may include a serum sample, a plasma sample, a urine sample, a liquor sample, a blood sample, a tear sample, a saliva sample, a vaginal swab, a stool sample, a viral particle, a protein, a cell supernatant, a cytokine, an immunoglobulin, and the like.

As used herein, the term “recipient cell” refers to at least one cell receiving information (biomaterial) from a donor cell, wherein the biomaterial can be EVs, soluble ligands internalized with their plasma membrane receptor or virus or by an infectious agent. The recipient cell includes, but is not limited to, a melanoma cell, a mesenchymal cell, blood cells, mesenchymal stromal cells, bone cells, muscle cells, epithelial cells, endothelial cells, immune cells, dendritic cells, somatic cells, germ cells, neuron and cells derived from various organs (such as brain, retina, pancreas, prostate, lungs, stomach, heart, spleen, kidney, thymus, cornea, bladder, oesophagus, colon and so forth).

As used herein, the term “donor cell” refers to a transformed cell, such as a cancer cell or infected cell. Furthermore, the donor cell can also be healthy cells communicating with transformed cell or other healthy recipient cells.

As used herein, the term “subject organism” refers to an organism that is subject to a disease or condition. The subject organism includes a unicellular or a multicellular organism, for example, a human, an animal, a plant, a fungus, a bacterium, an alga, a virus, and so forth.

As used herein, the term “multicellular organism” refers to a eukaryotic organism including more than one cell, such as a human, an animal, a plant, a fungus, an alga and the like.

As used herein, the term “epitope” refers to a portion of an antigen, for example a CD9, against which an antibody is produced and to which the antibody is directed for binding. Typically, epitopes are short amino-acid sequences, comprising a linear or non-linear sequence of about 5 to 8 amino acid residues.

As used herein, the term “monoclonal antibody (mAb)” refers to an antibody obtained from a population of substantially homogenous (or identical) immune cells. Generally, the identical immune cells are clones of a unique parent cell, for example, B-cell, T-cell, plasma cell, and so forth. The mAb have monovalent affinity for binding to the same epitope (in the extracellular part of the CD9 of an antigen) that is recognized by the mAb. Typically, the mAb have a weight in a range of 100-150 kDa.

As used herein, the term “cancer” refers to a disease or condition caused by cancerous cells (or carcinoma or carcinogenic cells or tumours), especially the malignant cancer/tumour cells. Moreover, the counterpart of the malignant cancer/tumour cells are benign tumour cells that do not cause cancer. More specifically, the cancerous cells possess a potential to invade their neighbouring cells and spread over different parts of a body of the subject organism.

As used herein, the term “cancer metastasis” refers to the regional growth of cancer, i.e. the potential of cancerous cells to spread from one part to another and in the process arranging for resources (from the host cell) supporting its growth.

As used herein, the term “virus” refers to a small infectious agent. Generally, the viruses are non-living organisms outside the subject (host) organism, however, they lead a normal reproductive live inside the subject (host) organism. Specifically, the virus replicates only inside the living cells of other organisms, such as animals, plants, humans, bacteria, and so forth.

As used herein, the term “budding off” refers to a process where a small part of the plasma membrane of the cell of a subject organism, such as a human, is released outside the cell. For instance, EVs are budding off from cells.

As used herein, the term “budding off” also refers to a process where a small part of the plasma membrane of the cell of a subject organism, such as a human, wraps a virus particle leading to a fusion of the virus and the human cell.

As used herein, the term “pharmaceutical composition” refers to a growth regulating agent that promotes recovery from a disease or condition. Typically, the pharmaceutical composition, includes but is not limited to, capsules, beads, granules, tablets, coated tablets, small tablet units, mini-tablets, bi-layered tablets, multi-layered tablets, tablet-in-tablet, powders, pills, pellets, micropellets, multiple unit pellet system (MUPS), sprinkles, syrups, suspensions, sachets, emulsions, dispersions, elixirs, solutions, liquids and concoctions. The pharmaceutical composition can be consumed orally or administered intravenously. However, other suitable route of administration can be employed, for example, such as transdermal, topical, parenteral, ocular, vaginal, rectal, buccal, lingual, intranasal and inhalation.

As used herein, the term “excipient” refers to a substance formulated alongside an active pharmaceutical ingredient (API) of a pharmaceutical composition. Typically, the excipients possess no medicinal properties; however, they may aid in lubricity, flowability, integration, taste, provide long-term stabilization, bulk up the formulation containing the potent API in small amounts and facilitate physiological absorption of the pharmaceutical composition amongst other properties.

As used herein, the term “adjuvant” refers to drugs of no or negligible pharmacological effect, but capable of increasing the efficacy and/or potency of other pharmacologically active drugs when administered together. Therefore, the adjuvant used must be of high purity and low toxicity for their use with pharmaceutical compositions.

As used herein, the term “carrier” refers to a substrate used in the process of delivery of a pharmaceutical composition at a target site. Typically, a pharmaceutical carrier serves to improve the selectivity, effectiveness and/or safe delivery of the pharmaceutical composition.

### DESCRIPTION OF EMBODIMENTS

The following detailed description illustrates embodiments of the present disclosure and ways in which they can be implemented. Although some modes of carrying out the present disclosure have been disclosed, those skilled in the art would recognise that other embodiments for carrying out or practising the present disclosure are also possible.

The present disclosure provides an antigen-binding fragment (Fab) and/or an aptamer that recognises the target epitope in an extracellular part of CD9, wherein the Fab and the aptamer bind directly to the extracellular part of the CD9, and wherein the Fab binds to the extracellular part of the CD9 at a similar location as that of the aptamer. Specifically, the antigen-binding fragment (indicated as ‘Fab’ hereafter) and/or the aptamers have high specificity and high affinity towards corresponding cells and/or EVs. More specifically, the Fab and/or the aptamer recognises particular epitopes in the extracellular part of the CD9. The recognition of the epitope is followed by binding of the Fab and/or the aptamer to a predefined location on the epitope. Moreover, the aptamers enable substantial recognition of a molecular targets, such as an epitope on the extracellular part of the CD9 as compared to the corresponding antibodies, thereby resulting in substantial binding of the aptamer to its molecular targets with high affinity and specificity. It is evident from the current literature that different epitopes may confer different properties and reactivities to the antibody. For example, different epitopes of the stem cell marker, prominin-1 (CD133) have different reactivities: the widely used AC133 antibody, which recognizes an epitope that is dependent on glycosylation, does not detect prominin-1 in adult human epithelia, that is instead detected by antibodies targeting different epitopes (Karbanova et al. J. Histochem. Cytochem. 2008).

Notably, the Fab and the aptamer are specific to a particular epitope and bind directly to the extracellular part of the CD9 at a similar location, i.e. the Fab binds to the extracellular part of the CD9 at a similar location as that of the aptamer or at a location nearly-adjacent (namely, in close vicinity). By the term “similar location” is meant that the binding of the Fab to the extracellular part of the CD9 is determined to be substantially similar to the binding of the aptamer to the extracellular part of the CD9. It may be appreciated that the Fab and the aptamer bind to a specific epitope at similar locations, such as a linear amino acid sequence of 3-12 amino acids, or conformational residues within 2- and/or 3-dimensional sequences recognised at separate locations on the extracellular part of the CD9. Likewise, the similar location of the binding of the Fab is identified adjacent or near the binding of the aptamer to the extracellular part of the CD9. Furthermore, binding of the Fab and the aptamer at similar location to inhibit the CD9 results in a conformational change in the CD9 to reflect distinct activation states associated with intercellular communications, adhesion endocytosis, signal transduction and cell-cell/cell-EV fusions. The bindings of the Fab and/or the aptamer affect CD9 dimerization and/or multimerization processes. Such activation states are reflected by one or more epitopes, whose expression reflects changes in the avidity and/or affinity of the associated CD9 to other biocomponents in the vicinity. The binding of the Fab and the aptamer at similar location on the extracellular part of the CD9 results in complete inhibition or neutralization of the CD9 on the cells and/or EVs. Consequently, the endocytosis of the EVs is inhibited and the nuclear transfer of the EV-derived cargo proteins is abolished. When the Fab and an aptamer are used together, the high specificity and affinity of the aptamers towards the targeted molecule saves any chance of failed inhibition (namely, effectively guarantees inhibition) of the targeted molecule by only the antibody or a fragment thereof. Additionally, the aptamers possess stable storage properties and elicit negligible or no immune response in the recipient cells. Beneficially, the aptamers are novel and synthesized based on the nucleotide and/or peptide sequence of the Fab or the target epitope in order to achieve a specific binding of the Fab at the target location.

In an example, the Fab may recognise mouse cells transfected with human CD9-containing cDNA construct. In another example, the aptamers MO-1 and MO-2 possess a high affinity for the cells and/or EVs, and target proteins, such as integrins, major histocompatibility complex (MHC), tumour necrosis factor (TNF), heat shock protein 70 (HSP70), intercellular adhesion molecule-1, CD9, CD13, CD26, CD63, CD81 and so forth, on the surface of the EVs as well as the transcription factors, growth factors, immunoglobulins, enzymes and receptor molecules on the surface of the antigen or recipient cells associated with cancer cells.

In an embodiment, the present disclosure provides the Fab and aptamer to recognise and bind to the target epitope in the extracellular part of CD9. Beneficially, similar nucleotide and/or peptide sequence of the Fab and the aptamer results in increased binding affinity and specificity. Fab and aptamers specifically bind at specific sites of the target epitope, such as that of cancer cells, bacteria, virus, and the like, and inhibits the activity of the target epitope. Notably, due to similar sequence of the Fab and the aptamer, the two have similar binding sites, for example nearly-adjacent to each other.

In another embodiment, the present disclosure provides the aptamer to recognise and bind to the target epitope in the extracellular part of CD9. Beneficially, similar nucleotide and/or peptide sequence of the Fab and the aptamer results in increased binding affinity and specificity. Aptamers specifically bind to molecules, such as cancer cells, bacteria, virus, and the like, through non-covalent interactions, such as electrostatic interactions, hydrophobic interactions, and the like, or their complementary shapes. The two or three-dimensional structure of the aptamers substantially increases the recognition surface area thereby enabling interaction with the target epitope. Consequently, binding of the aptamer at the target epitope inhibits the activity of the target epitope without provoking undesirable side-effects. Furthermore, aptamers function well in physiological conditions, and have a high shelf-life, while still chemically synthesized in just a few minutes and in a cost-effective manner. Furthermore, due to their small size, aptamers can penetrate membranes and target antigens of smaller sizes. Moreover, using Fab and aptamers together to target the epitopes is more beneficial in terms of enhancing each other's activities thereby exhibiting a synergistic effect.

Optionally, the Fab is a fragment of a monoclonal antibody and has a size of approximately 40-60 kDa, 45-55 kDa, 48-52 kDa or 50 kDa, and wherein the monoclonal antibody is at least one of an anti-human CD9 and an anti-CD9. Specifically, the Fab is derived from a monoclonal Ab (indicated as ‘mAb’ hereafter). More specifically, the mAb, upon digestion, provides fragment of mAb (Fab). Typically, the Fab is composed of a constant and a variable domain of each of the heavy and the light chain. The variable domain indicates a region of an antibody which contains an antigen-binding site (or a paratope). The antigen-binding site comprises a set of complementarity-determining regions (CDR), that binds an epitope on the antigen. Furthermore, the variable domain demonstrates modifications in sequence, indicated as CDR-1, CDR-2 and CDR-3, involved in recognizing antigen. Notably, a typical mAb has size in a range of 100-150 kDa, and a fragment of the mAb is of a significantly lower size. In an example, the Fab is approximately 40-60 kDa. By the term “approximately” is meant that the size of the Fab may be in a range of 30 to 70 kDa under non-reduced conditions. The approximate lower size of the kDa range may include 35, 36, 37, 38, 39, 40, 41, 42, 43, 44, 45 kDa, etc. The approximate higher size of the kDa range may include for example 50, 55, 56, 57, 58, 59, 60, 61, 62, 63, 64, 65, kDa, etc. Ranges including all possible variations of these values are also disclosed herein. In another example, the Fab has a size of 50 kDa. The scFv (single-chain variable fragment) and Fv (variable fragment) fragments derived thereof might also be applicable. Genetic engineering antibodies with monovalent binding site to CD9 as well.

Furthermore, the mAb is at least one of the anti-human CD9 and the anti-CD9. The term “anti” as used herein refers to a complement of, for example CD9 proteins present on the EVs and/or recipient cells. The anti-CD9 monoclonal antibodies (anti-CD9 mAb) are the monoclonal antibodies that are binding specifically to CD9 proteins. The anti-CD9 mAb bind an epitope on the antigen and inhibit the transendothelial migration of the antigen. However, anti-CD9 mAb enhances the uptake of EVs by the recipient cells. Silencing of CD9 both in the EVs or the recipient cells strongly reduces the endocytosis of EVs by the recipient cells. The anti-human CD9 Fab recognise the human CD9 cell-surface antigens. In an embodiment, the anti-CD9 mAb or CD9 Fab may recognise the human CD9 protein in its native form or non-reduced form. Moreover, specificity of an antibody towards an antigen depends on the modifications in sequence in that region. In an example, the mAbs specifically recognise an epitope specific to a cancer cell and binds only to cancer cell-specific antigens presenting the epitope; and serve to induce an immune response against the target cancer cell. Alternatively, bispecific monoclonal antibodies, when in operation, can target two epitopes in a same analyte or different analytes. In another example, the mAbs may also bind to a conjugate or effector cell that enhances the efficacy of the target cell.

Optionally, the monoclonal antibody is an antibody derived from at least one of antigens including TSPAN29, DRAP-27, BTCC-1, motility related protein-1, leukocyte antigen MIC3, 5H9, MRP-1 and BA-2/P24. It will be appreciated that TSPAN29, DRAP-27, BTCC-1, motility related protein-1, MRP-1 and MIC3 are the other nomenclatures for the CD9. Moreover, the CD9 is referred to as the ‘motility related antigen, MRP-1’ when related with cell motility and the cancer metastasis. Furthermore, a nucleotide sequence of cDNA encoding the 5H9 antigen is the same as that of a nucleotide sequence encoding the human CD9 cDNA.

Optionally, the fragment of the monoclonal antibody (CD9 Fab) is derived from a 5H9 anti-CD9 monoclonal antibody, optionally wherein the fragment is derived by digestion of the antibody with papain. The CD9, also known as 5H9 antigen, is encoded by the CD9 gene in humans. Therefore, a fragment of the mAb targeted at the 5H9 antigen is derived from the 5H9 anti-CD9 monoclonal antibody (indicated as ‘CD9 Fab’ hereafter). Notably, the 5H9 CD9 Fab blocks the uptake of EVs by the recipient cells and thereby the nuclear transfer of the EV-derived cargo proteins. Moreover, the CD9 Fab is derived by digestion of the mAb with papain. It will be appreciated that papain is a protease enzyme that breaks peptide bonds. Specifically, papain (and/or pepsin) digestion of the anti-CD9 monoclonal antibody results in two identical halves of the mAb, each composed of a heavy chain and a light chain, connected by two disulfide bonds. Each heavy chain is composed of an antigen-binding fragment (Fab) and a crystalline fragment (Fc). In other words, papain cleaves the Fc (crystallizable) portion of the mAb from the Fab.

In an exemplary embodiment, the process of generating the CD9 Fab comprises incubating the full-length monoclonal antibody with a proteolytic enzyme, such as papain, immobilized on agarose resin (or papain-immobilized agarose resin) for a predefined period of time, such as in a range of 2 to 3 hours, at a predefined temperature, for example 37° C. The digested antibody is collected by centrifugation (5000×g for 1 minute) and the flow through containing the antibody is eluted in a new tube. The fragment crystalline (Fc) of the digested antibody is subsequently removed by centrifugation (1000×g for 10 minutes), for example by using NAb Protein A Plus Spin Column. Consequently, the centrifugation results in generation of Fab fragments in the flow through. Furthermore, anti-CD9 monoclonal antibody, having a size of approximately 150 kDa, when digested with papain yields three fragments, i.e. two CD9 Fab fragments having size of approximately 50 kDa each and one Fc fragment having size of approximately 50 kDa.

Optionally, other enzymatic techniques of production of Fab fragments or fragments of the invention are valuable. For instance, enzymatic digestion can be performed with ficin, pepsin, trypsin, instead of papain.

Optionally other chemical techniques of production of Fab fragments or fragments of the invention are valuable. Chemical compounds including cross-linkers can be used alone or in combination with proteolytic enzymes.

Optionally, production of recombinant Fab or fragments of the invention is possible using a wide range of expression systems such as bacteria, yeast, insect and mammalian cells.

Optionally, Fab fragment or fragments thereof can be derived from any immunoglobulins notably IgG or IgM and their subtypes.

Optionally, Fab fragment or fragments thereof can be purified by other methods that consist of clarification, capture chromatography, polishing chromatography, electrophoresis gel and ultrafiltration/diafiltration. Size-exclusion chromatography and immobilized metal affinity chromatography (in case of His-tagged antibody and fragment thereof) can be used.

Optionally, the 5H9 anti-CD9 monoclonal antibody fragment binds directly to the extracellular part of the CD9. Typically, the CD9 Fab is employed in recognizing the epitopes in the extracellular part of the CD9 of the antigen, and optionally of the recipient cells. The CD9 Fab binds to the extracellular part of the CD9, thereby reducing the ability of CD9 to mediate the intercellular communication. Specifically, the CD9 Fab is highly reactive and recognises a band in a range of 24 kDa to 27 kDa corresponding to the CD9 and undergoes immunoprecipitation. In an example, the CD9 Fab may recognise mice cells transfected with human CD9-containing cDNA construct. In another example, the CD9 Fab may be employed for the treatment of different types of diseases, such as cancer, cancer metastasis and infections, by means of inhibiting the cell surface receptors proteins, such as CD9. Notably, the CD9 is associated with multiple types of cancer, including, but not limited to, non-T-cell acute lymphoblastic leukemia, chronic lymphocytic leukemia, acute myeloblastic leukemia, cancer metastasis, carcinoma metastasis, breast carcinoma, lung carcinoma and colon carcinoma. Specifically, the CD9 is involved in suppression of cancer motility and cancer metastasis, therefore, the CD9 is a potential therapeutic target for the diagnosis and prevention of cancer metastasis. A 5H9 anti-CD9 monoclonal antibody fragment (CD9 Fab) specifically inhibits the intercellular migration of melanoma cells. However, the anti-CD9 monoclonal antibody (Ab) may enhance the nuclear uptake of EVs in a recipient cell. Moreover, silencing of CD9, both in the EVs and the recipient cells strongly decreases the endocytosis of EVs by the recipient cells, and furthermore inhibits the nuclear transfer of the EV-derived proteins and nucleic acids in the recipient cells.

Optionally, the aptamer is made of DNA or RNA and the aptamer is a CD9 aptamer. The aptamer contains short strands of oligonucleotides, such as a DNA, a RNA or nucleotide acids analogues (represented as ‘XNA’). The nucleic acid aptamers bind to various molecular targets, such as the CD9, proteins, nucleic acids, cells, tissues and so forth. Furthermore, the nucleic acid aptamers employ at least one of: non-covalent interactions, electrostatic interactions, hydrophobic interactions, and their complementary shapes. The DNA and RNA aptamers show robust binding affinities for various molecular targets and exhibit no systematic differences in their activities. However, DNA aptamers exhibit greater intrinsic chemical stability. In an example, the DNA/RNA aptamers may include, but are not limited to siRNA, miniRNA, microRNa, IncRNA, antisense oligonucleotides, single-stranded DNA and cDNA. The peptide aptamers consist of one or more short variable peptide domains, attached at both ends to a protein scaffold. The peptide aptamers can bind to the cellular proteins and exert substantial biological effects by interfering with the normal protein interactions with the targeted molecules.

In an embodiment, the aptamer is a CD9 aptamer. The CD9 aptamers specifically target the CD9s on the surface of the EVs. The CD9 aptamers can be agonists or antagonists of the CD9s. The CD9 aptamers can be of any chemical or biochemical structure and nature.

Optionally, the antigen-binding (Fab) and/or the aptamer inhibits the intercellular communication in multicellular organisms by interfering with at least one of: the endocytosis of extracellular membrane vesicles (EVs); the fusion of EVs with cells; and the intracellular transport of endocytosed biomaterials in recipient cells. It will be appreciated that the cells interact with each other and their surroundings to mediate the transfer of biomolecules in and out of the cell. Such interaction of two or more cells occurs through various intercellular communication strategies, including but not limited to cell-cell contact, soluble molecules, quorum sensing, extracellular vesicles, and so forth. The Fab and/or the aptamers inhibit the intercellular communication in multicellular organisms. Furthermore, the EVs are taken up by the recipient cells through the process of endocytosis. The endocytosis of EVs is mediated by the various mechanisms involving cytoplasmic proteins and cell surface proteins, such as CD9, which is also present on the surface of EVs. In this process, the CD9 is involved in the reorganization of the biocomponents, such as proteins and lipids, of the plasma membrane into a specific tetraspanin web or other membrane microdomains that regulates the interaction with EVs and promotes their endocytosis. Furthermore, the CD9 acts as a scaffold in the regulation of adhesion molecules at the immune synapse and T lymphocyte activation.

Notably, the endocytosis of EVs plays a major role in physiology and pathology. Moreover, the Fab and/or the aptamers interferes with the endocytosis of the EVs by targeting the cell surface protein CD9. Endocytosis of EVs is followed by the fusion of EVs with the recipient cells. The CD9 has been described to play a role in cellular adhesion and fusion. Moreover, the Fab and/or the aptamers further interfere with these processes between the EVs and the recipient cell by targeting the CD9. The inhibition of the CD9 by the biological molecular assay reduces the subsequent transfer of the EV-derived cargo proteins to the recipient cell, and the intracellular transport of endocytosed biomaterials in recipient cells. Specifically, the Fab and/or the aptamers interfere with the nuclear transfer of genetic material from the endocytic pathway and/or cytoplasm into the nucleus of the recipient cells. In an example, the CD9 Fab interferes with the uptake of EVs by recipient cells, such as melanoma cells and MSCs, as well as with the nuclear transfer of the EV-derived cargo proteins as a result of the inhibition of the endocytosis of EVs. Furthermore, silencing of the CD9s in the EVs, the recipient cells, or both, impedes, reduces or limits EV endocytosis.

Optionally, the antigen-binding fragment (Fab) and/or the aptamers are usable in at least one of medicine, veterinary medicine, agriculture and insecticide. The Fab and/or the aptamers are directed at identifying and quantifying an antigen responsible for onset and progression of a disease or condition. Therefore, such Fab and/or the aptamers may be used to target various domains, such as at least one of medicine, veterinary medicine, agriculture and insecticide, associated with maintaining healthy conditions of living organisms. It will be appreciated that despite variations in the amino acid sequence, the CD9 is well conserved, about 90%, between human, mice and rat species. Therefore, the Fab and/or the aptamers may be employed to monitor effects of different medicines and different concentrations thereof in different organisms, such as mice and rat species, before trying the medicines and different concentrations on humans. Similarly, effects of agricultural preparations may be tried and tested for a suitable concentration corresponding to a species of plant, crop, and so forth. Furthermore, insecticidal preparations may be targeted against living organisms like plants, animals and humans, as well as and non-living things such as wooden furniture, paper, sewer and so forth, to control the growth of pathogens on the target organisms and articles, such as furniture, books and the like.

More optionally, the antigen-binding fragment (Fab) and/or the aptamer is usable as a medicament. It will be appreciated that the medicament is an agent, such as an active pharmaceutical ingredient, that may promote recovery from a disease or condition. In an embodiment, the medicament may comprise a strain of a pathogen, which is a strain present in the subject organism and is responsible for the onset and progression of the disease or condition. Typically, the medicament is administered to the subject organism, for example orally, intravenously, transdermal or any other route of administration.

Optionally, the antigen-binding fragment (Fab) and/or the aptamer is for use in the treatment and/or prevention of a disease or condition in which the endocytosis of extracellular membrane vesicles (EVs) is implicated, optionally wherein the subject is a human. Notably, various diseases occur due to at least one of: the endocytosis of extracellular membrane vesicles (EVs), the fusion of EVs with cells and the intracellular transport of endocytosed biomaterials in the recipient cells. Optionally, the disease or condition includes at least one of cancer, cancer metastasis and infection. Without being bound to theory, it will be appreciated that in cancer disease, the EVs promote pro-angiogenic events and alter the surrounding cellular components as well as extracellular matrix to develop the pre-metastatic niche. Notably, the production of EVs is deregulated in human diseases, such as in cancer, and thus, the EV-derived cargo proteins serve as potential biomarkers. Furthermore, the EVs can be engineered for the selective therapeutic delivery of biomacromolecules.

Furthermore, the CD9 plays an important role in causing cancer, cancer metastasis and infections. The Fab and/or the aptamer inhibits the numerous properties of the CD9, including cell adhesion and fusion, motility and growth, to enable early diagnosis and treatment of cancer, cancer metastasis and infection in the subject organisms. Furthermore, the Fab and/or the aptamer allows for monitoring the effectiveness of a therapeutic intervention and standardizing a protocol employed by the therapeutic intervention against the treatment of such diseases in the subject organisms. More optionally, the subject organism is a human. Being a multicellular organism, humans are more susceptible to pathological attack as the cells interact with each other as well as their environment to perform specialized functions. Moreover, the surface of human cells exhibits the CD9 similar to the CD9 of the EVs. Consequently, the Fab and/or the aptamer are also targeted at the CD9 present at the surface of the EVs, such as a carcinoma, as well as the CD9 present on the human cells, thereby preventing or treating the disease including the endocytosis of EVs in humans.

Optionally, the infection is caused by a virus. Typically, the infection is caused due to various pathogens including, but not limited to, a bacterium, a virus, a fungus, a protozoan, and so forth. Specifically, the infection caused by a virus, namely a viral infection, involves the nuclear transport of endocytosed viral biomaterials to the recipient cells. More optionally, the virus is an enveloped virus, optionally the virus is HIV-1. It will be appreciated that the enveloped virus comprises of an outer cover or envelope, that usually includes CD9. Moreover, CD9 is present on the plasma membrane of the recipient cell near the HIV virus entry site. Typically, the envelope is generated by biocomponents of the subject organism around the virus by budding-off of the plasma membrane of the cell of the subject organism around the endocytosed virus. Such fusion of virus and human cell initiates the intracellular transport of endocytosed biomaterials in the human cells. Furthermore optionally, the virus is HIV-1. The HIV-1 virus attacks immune system of the subject organism, for example, such as a CD-4+ cell composing the immune system of the humans. The HIV-1 virus causes a viral infection, namely AIDS, which severely damages the immune response of the subject organism, specifically humans. Specifically, like the cancerous cells, virus-infected cells also secrete EVs, thus large number of EVs is secreted during viral infection. More specifically, viral particles, proteins and RNA, are transferred from a virus-infected cell into nucleus of the recipient cell via the EVs. Nonetheless, the viral cells also secrete virions, along with the EVs, for transferring the viral biocomponents into the recipient cells. Moreover, the treatment or prevention of the infection requires directly targeting the pathogen, i.e. the bacteria, the virus, the fungus and/or the protozoan.

Optionally, the antigen-binding fragment (Fab) and/or the aptamer is used to impede the release of virus from infected donor cells. Membrane partition and remodelling are involved in the virus release in which CD9 (or other tetraspanin proteins) forms enriched membrane areas where the virus buds (Dahmane S et al. 2019, Nanoscale).

Optionally, the disease is a neurodegenerative disease. Specifically, the neurodegenerative diseases target the neurons or nerve cells in the brain. Typically, the neurodegenerative diseases include Alzheimer's disease, Parkinson's disease, Creutzfeldt-Jakob disease (CLD), Huntington's disease, bovine spongiform encephalopathy, multiple sclerosis disease, and so on. It will be appreciated that the EV's of the subject organism contain a protein associated with the neurodegenerative disease. Furthermore, the EVs of the subject organism, such as a human, contain various proteins associated with the neurodegenerative disease. More optionally, the protein associated with the neurodegenerative disease includes at least one of: a prion protein, a beta-amyloid protein, an alpha-synuclein protein and a tau protein. The pathogen, such as the HIV-1 virus, produces prions, an infectious particle responsible for the transmissible neurodegenerative disease, including Creutzfeldt-Jakob disease (CLD) in humans and the bovine spongiform encephalopathy in animals, specifically cattle. Furthermore, the amyloid precursor protein (APP), an integral membrane protein produced by the EVs, is associated with Alzheimer's disease. Specifically, the amyloid precursor protein (APP) generates the beta-amyloid protein upon proteolysis. Furthermore, the tau protein is associated with Alzheimer's disease, Parkinson's disease, progressive supranuclear palsy (PSP), cortico-basal degeneration (CBD), dementia, and so forth. Beneficially, the tau proteins are potential biomarkers for differential diagnosis and monitoring of therapeutic interventions for Alzheimer's disease and dementia. Similarly, the alpha-synuclein protein is a neuronal protein associated with several neurodegenerative diseases, including a familial form of Parkinson's disease and dementia.

Optionally, the disease is Alzheimer's disease, Parkinson's disease, ventricular hypertrophy, type I diabetes, type II diabetes, a kidney disease, macular degeneration or a lung disease. It will be appreciated that the EVs-mediated intercellular communication is also implicated in various diseases including, but not limited to, a neurodegenerative disease, Alzheimer's disease, Parkinson's disease, ventricular hypertrophy, type I diabetes, type II diabetes, kidney disease, macular degeneration and/or lung disease. Moreover, the ventricular hypertrophy, the type I diabetes and/or type II diabetes, the kidney disease, the macular degeneration and the lung disease are associated with specific cell types, such as a cardiac cell, a pancreatic cell, a renal cell, a cone cell and an alveolar macrophage cell.

The present disclosure provides a pharmaceutical composition comprising the antigen-binding fragment (Fab) and/or the aptamer, and at least one pharmaceutically acceptable excipient, adjuvant or carrier. It will be appreciated that the pharmaceutical composition comprises only the pharmaceutically acceptable compounds, such as excipients, adjuvants, carriers along with suitable Fab and/or aptamers to target the CD9 on the EVs or on the cells of the subject organism. In an embodiment, the pharmaceutically acceptable excipient is selected from a group consisting of one or more water-soluble agents, such as lactose, mannitol, calcium sulphate, dextrin, dextrates, dextrose, sucrose, povidone and the like; water-dispersible diluent, such as microcrystalline cellulose, powdered cellulose, starch (corn starch, pregelatinized starch), clay or clay minerals (kaolin, bentonite, attapulgite); glindant, such as calcium carbonate, magnesium stearate, stearic acid and the like; anti-adherent, such as talk, titanium dioxide, red and yellow iron oxide, sodium lauryl sulfate and the like; lubricant, such as silicon dioxide, hydrogenated vegetable oil and the like, preservative, such as butylated hydroxyanisole and the like; suspending agent, such as sodium carboxymethylcellulose, methylcellulose and the like; and so forth. Moreover, the pharmaceutically acceptable adjuvant may be selected from a group consisting of buffering agents, such as citric acid, sodium citrate and the like; preservatives, such as sodium benzoate and the like; anti-cracking agents, such as colloidal silicon dioxide and the like; flavours to mask bitter taste; suspending agents, such as xanthan gum, carrageenan and the like; antifoaming agents, such as simethicone and the like; and colouring agents, such as ferric oxide and the like.

Furthermore, the pharmaceutically acceptable carriers may be present in the pharmaceutical composition in a co-milled state, wherein the active pharmaceutical ingredient (API) of a pharmaceutical composition and the pharmaceutically acceptable carrier are milled to form a mixture or the active pharmaceutical ingredient (API) of a pharmaceutical composition and the pharmaceutically acceptable carrier are extensively mixed prior to milling. The milling of the ingredients is conducted using a suitable milling equipment, known to a person skilled in the art. Optionally, the ratio of the pharmaceutically acceptable carrier in the pharmaceutical composition ranges from about 3:1 to about 1:10, more preferably, about 1:1 to about 1:5, in particular about 1:2 to about 1:3, based on parts per weight. The pharmaceutically acceptable carrier may include but not limited to hydroxypropylmethylcellulose (HPMC), hydroxypropylcellulose (HPC), polyvinylpyrrolidone (PVP) (such as povidone K12), povidone K30 and microcrystalline cellulose (MCC).

Optionally, the pharmaceutical composition is for use in the treatment and/or prevention of a disease or condition selected from cancer, cancer metastasis, infection, a neurodegenerative disease, Alzheimer's disease, Parkinson's disease, ventricular hypertrophy, type I diabetes, type II diabetes, kidney disease, macular degeneration and lung disease. In this regard, the pharmaceutical composition is targeted at cancer cells, neurons, cardiac cells, pancreatic cells, renal cells, rods and cones of the eyes, and alveolar cells of the subject organism.

Optionally, the antigen-binding fragment (Fab), the aptamer or the pharmaceutical composition is usable when the patient is further administered one or more therapeutic agents or when the Fab and/or the aptamer is provided in combination with one or more therapeutic agents. Notably, when the Fab, the aptamer or the pharmaceutical composition is combined with other approaches, such as molecular evolution techniques and/or one or more therapeutic agents, such combination may be used to directly and successfully target molecules, such as cancer cells and pathogenic cells, and lead to a new modality in diagnosis and treatment of diseases by inhibiting the intercellular communication within the disease cell niche or distal microenvironment.

Optionally, the pharmaceutical composition is for use in combination with one or more therapeutic agents for the treatment and/or prevention of the disease or condition. More optionally, the one or more therapeutic agents may correspond to the treatment and/or prevention of the disease itself or to the side effects of such disease. For example, a pharmaceutical composition to treat the ventricular hypertrophy may be usable with a blood thinning pharmaceutical composition, such as an ecosprin, which treats the disease as well as the associated symptoms of the disease.

The present disclosure also provides a biological molecular assay that recognises the target epitope in the extracellular part of CD9, wherein the biological molecular assay includes use of the antigen-binding fragment (Fab) and/or the aptamer, and wherein the Fab and the aptamer bind directly to the extracellular part of the CD9, and wherein the Fab binds to the extracellular part of the CD9 at a similar location as that of the aptamer. Notably, the epitopes are located in the extracellular part, specifically the larger extracellular part of the CD9. The biological molecular assay recognises the target epitope in the extracellular part of a CD9. The Fab and the aptamer recognize a particular epitope on the extracellular part of the CD9. The recognition of the epitope is followed by binding of the Fab and/or the aptamer to a predefined location on the epitope. The locations for binding of the Fab and the aptamer are mutually similar. When the Fab and an aptamer are used together, the high specificity and affinity of the aptamers towards the targeted molecule saves any chance of failed inhibition (namely, effectively guarantees inhibition) of the targeted molecule by only the antibody or a fragment thereof. Furthermore, the biological molecular assay recognizes both the conformation-dependent epitopes, as well as the conformation-independent epitopes.

Optionally, the Fab is a fragment of a monoclonal antibody and has a size of approximately 40-60 kDa, 45-55 kDa, 48-52 kDa or 50 kDa, and wherein the monoclonal antibody is at least one of an anti-human CD9 and an anti-CD9. Specifically, upon papain digestion of a mAb, more specifically of an anti-human CD9 and an anti-CD9, the fragment thereof has a size in a range of 40 kDa to 60 kDa, preferably 45 kDa to 55 kDa, more preferably 48 kDa to 52 kDa, still more preferably of 50 kDa.

Optionally, a 5H9 anti-CD9 monoclonal antibody fragment binds directly to the extracellular part of the CD9. Typically, the mAb and the Fab derived thereof are employed in the biological molecular assay to recognize the epitopes in the extracellular part of the CD9 of the antigen. The 5H9 anti-CD9 monoclonal antibody fragment binds to the extracellular part of the CD9, thereby reducing the intercellular communications.

Optionally, the 5H9 anti-CD9 monoclonal antibody and the fragment thereof are highly reactive. The 5H9 anti-CD9 monoclonal antibody fragment has a specific pattern of immunofluorescence staining of monolayers of cells. The CD9 Fab recognizes a band in a range of 24-27 kDa corresponding to the CD9 and undergoes immunoprecipitation. In an example, the 5H9 anti-CD9 monoclonal antibody and the fragment thereof may recognize mice cells transfected with human CD9-containing cDNA construct. In another example, the 5H9 anti-CD9 monoclonal antibody and the fragment thereof may be employed for the treatment of different types of diseases, such as cancer, cancer metastasis and infections, by means of inhibiting the cell surface receptors proteins, such as CD9. Notably, when combined with other approaches, such as molecular evolution techniques, aptamers and the like, the monoclonal antibodies and fragments thereof may directly and successfully target molecules, such as cancer cells, and lead to a new modality in diagnosis and treatment of diseases by inhibiting the intercellular communication within the disease cell niche.

Optionally, the biological molecular assay inhibits an intercellular communication in multicellular organisms by interfering with at least one of: an endocytosis of extracellular membrane vesicles (EVs); a fusion of EVs with cells; and an intracellular transport of endocytosed biomaterials in recipient cells. It will be appreciated that the cells interact with each other and their surroundings to mediate the transfer biomolecules in and out of the cell. Such interaction of two or more cells occurs through various intercellular communication strategies, including but not limited to cell-cell contact, soluble molecules, quorum sensing, extracellular membrane vesicles (EVs), and so forth. The biological molecular assay, in operation, employs the Fab and the aptamers to inhibit the intercellular communication in multicellular organisms. It will be appreciated that the multicellular organisms are eukaryotic organisms including more than one cell, such as humans, animals, plants, fungi, algae and the like. Furthermore, the EVs are taken up by the recipient cells through the process of endocytosis. The endocytosis of EVs is mediated by the various cytoplasmic proteins and cell surface proteins, such as CD9, present on the surface of cells as well as EVs. In this process, the CD9 is involved in the reorganization of the biocomponents, such as proteins and lipids, of the plasma membrane into a specific tetraspanin web or other membrane microdomain that regulates the interaction with EVs and promotes their endocytosis. Furthermore, the CD9 acts as a scaffold in the regulation of adhesion molecules at the immune synapse and T lymphocyte activation.

Optionally, the endocytosis of EVs play a major role in physiology and pathology. Moreover, the biological molecular assay interferes with the endocytosis of the EVs by targeting the cell surface protein CD9. After some duration, the EVs fuse with the recipient cells, wherein such fusion is mediated by the cell surface proteins, including CD9, and so forth. The CD9 is a fusion protein that is associated with cell adhesion, motility and fusion. In such an example, the biological molecular assay further interferes with the fusion of the EVs and the recipient cell by targeting the CD9. The inhibition of the CD9 by the biological molecular assay reduces the transfer of the EV-derived cargo proteins to the recipient cell, and the intracellular transport of endocytosed biomaterials in recipient cells, specifically the transfer of genetic material from the cytoplasm into the nucleus of the recipient cells. In this regard, the CD9 Fab employed by the biological molecular assay interferes with the uptake of EVs by recipient cells, such as melanoma cells and MSCs, as well as with the nuclear transfer of the EV-derived cargo proteins as a result of the inhibition of the endocytosis of EVs. Furthermore, silencing of the CD9s in the EVs, the recipient cells, or both, impedes, limits or reduces EV endocytosis.

Optionally, the biological molecular assay is usable in at least one of: a medicine, veterinary medicine, agriculture or as an insecticide. The biological molecular assay is directed at identifying and quantifying a pathogen or a biomolecule responsible for onset and progression of the disease or condition. Therefore, such biological molecular assay may be employed by various domains, such as medicine, veterinary medicine, agriculture, and insecticides, associated with maintaining healthy conditions of living or non-living things. The biological molecular assay can be employed to monitor effects of different medicines at different concentrations thereof in different organisms, such as mice and rat species, before trying the medicines and different concentrations thereof on humans. Similarly, effects of veterinary medicines and agricultural preparations may be tried and tested for a suitable concentration corresponding to a species of animals and plant respectively. Furthermore, insecticides may target insects dwelling on living organisms like plants, animals and humans, as well as on non-living things such as wooden furniture, paper, sewer and so forth. In such instance, the biological molecular assays can be used to prepare insecticides against the pathogenic species that can help control the growth of such pathogens on the target organisms and articles, such as furniture, books and the like.

Optionally, the biological molecular assay is useable, in operation, in combination with one or more therapeutic agents for treatment or prevention of the disease. The one or more therapeutic agents include a pharmaceutical composition, a therapy, or a combination thereof.

The pharmaceutical compositions of the invention may be in the form of a capsule, a bead, a granule, a tablet, a powder, a pellet, a syrup, a suspension, a concoction, and so on. The therapy may include radiation therapy, chemotherapy, laser therapy, anti-retroviral therapy (ARV) and so forth. The subject organism may be provided with the pharmaceutical composition and the therapy in constant or changing doses and for a predefined period of time. For example, the dose of the pharmaceutical composition may be 250 Units for the first 3 months, 500 Units for the next 3 months and 100 Units for 1 year in addition to radiation therapy using high energy X-rays at a dose of 60 Gray (Gy) for 2 hours thrice a month.

Optionally, the biological molecular assay is usable in the treatment and/or prevention of a disease or condition in which the endocytosis of extracellular vesicles is implicated, optionally wherein the subject is a human.

Furthermore, the pharmaceutical composition is useable, in operation, to implement the biological molecular assay. The pharmaceutical composition can be used to implement the biological molecular assay for treatment and/or prevention of the disease or condition. The biological molecular assay can be used in combination with one or more pharmaceutical compositions in different doses for prevention and/or treatment of the disease or condition at various stages. It will be appreciated that diseases diagnosed at an earlier stage may require lower dosage of the pharmaceutical composition as compared to the diseases diagnosed at later or higher stage of pathology.

In an exemplary implementation, the 5H9 anti-CD9 monoclonal antibody (CD9 Ab) is produced by fusing a myeloma cell line with spleen cells obtained from BALB/c J mice after immunization with the erythroleukemia cell line K562 and developed hybridoma producing anti5H9 by limiting dilution method. Furthermore, a FEMX-I cell line is derived from a lymph node metastasis of a patient with malignant melanoma. A human A375 melanoma cell line and a human C8161 melanoma cell line is also obtained. Cell lines are cultured in a growth medium comprising 10% fetal bovine serum, 2 mM L-glutamine, 100 U/mL penicillin and 100 μg/mL streptomycin. Human bone marrow-derived MSCs are isolated from bone marrow aspirates from normal adult donors. The MSCs and the FEMX-I cells express ectopically CD9-green fluorescent protein (GFP) fusion protein, and thus are used to produce fluorescent EVs useful in analysing the biological molecular assay.

Furthermore, the EVs are enriched by differential centrifugation from 72 hour-conditioned media of engineered FEMX-I cells and MSCs expressing CD9-GFP. The conditioned medium is centrifuged at 10,000×g for 30 min at 4° C., and the resulting supernatant centrifuged at 200,000×g for 60 min at 4° C. The pellet was resuspended in 200 μl phosphate-buffered saline (PBS). To determine the EV concentration, a 488 nm solid-state laser is used illuminate the EVs being injected, by continuous flow, into the sample chamber of an optical unit, such as a Nanosight LM10 unit. The calculated EV concentration was an average of six 30-sec video recordings. The average size of EVs produced by FEMX-I cells and MSCs is 123 nm and 114 nm, respectively. Subsequently, the cells are incubated with various concentrations of CD9-GFP+ EVs, for example 5×10⁷ particles/ml (0.075 μg/ml), 2.5×10⁸ particles/ml (0.375 μg/ml) or 1×10⁹ particles/ml (1.5 μg/ml), for 5 hours at 37° C.

In an embodiment, for analysis purposes, the cells are fixed, for example in 4% paraformaldehyde, and permeabilized, for example with 0.2% Tween 20 in PBS, for 15 min each at 37° C. The cells are subsequently blocked, for example with 1% BSA in PBS, and incubated with mouse anti-SUN2 Ab for 60 min each at RT. The cells are washed twice with PBS and incubated with tetramethylrhodamine (TRITC)-conjugated anti-mouse IgG or Cy5-conjugated anti-mouse IgG secondary antibodies for 30 min, and again washed with PBS prior to observation using confocal laser scanning microscopy (CLSM). Optionally, the CLSM employs a Nikon A1R+ inverted confocal microscope, preferably with, but not limited to a 60× Apo-TIRF oil-immersion objective and a numerical aperture of 1.49, high pixel resolution, such as 512×512 or 1024×1024. A 561 nm and a 638 nm solid-state laser is used to excite TRITC and Cy5, respectively, and corresponding fluorescence emissions were collected using 570 nm to 620 nm, and 662 nm to 737 nm long pass filters. Raw images are processed using Fiji. It will be appreciated that data is calculated collectively over multiple optical sections through the cell, such as 21 optical sections of 0.4 μm for cancer cells and 21 optical sections of 0.2 μm for MSCs, and any observed GFP fluorescent signal is counted as EV-derived biomaterials.

According to an embodiment, to determine the value of nucleoplasm GFP fluorescence for each cell, a region of interest (ROI) along the nucleus of each cell is selected and the nuclear fluorescent materials covered in such selection are counted. According to another embodiment, a region of interest (ROI) may be selected along the cytoplasm of each cell, and by using cell border as a guide and excluding the nucleus, the cytoplasmic fluorescent materials covered in such selection are counted.

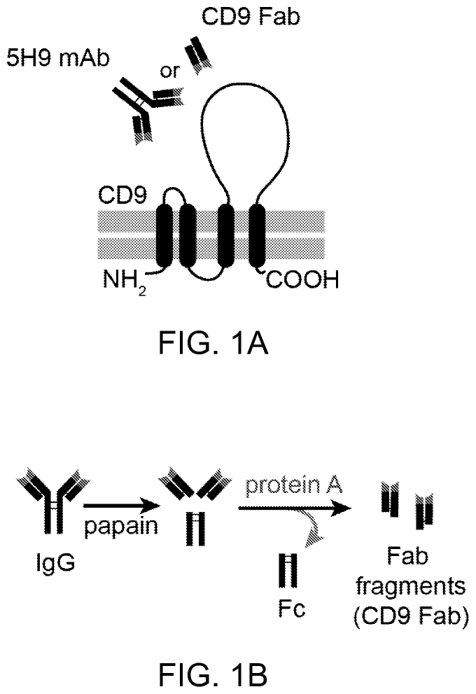

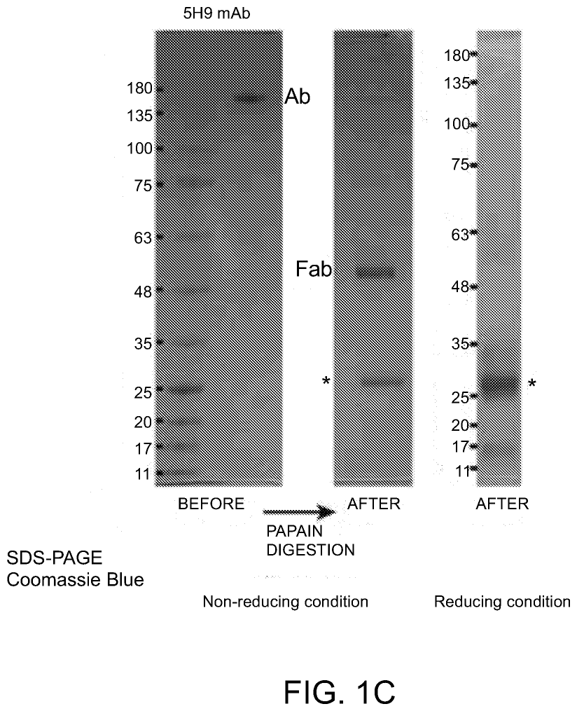

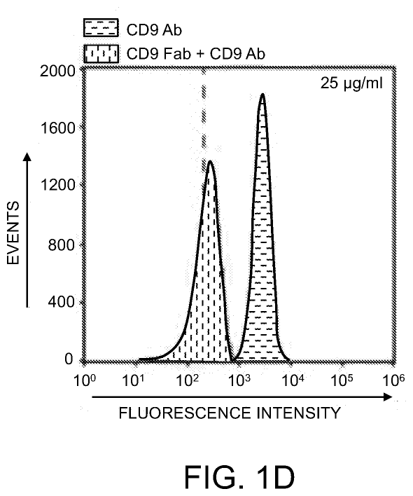

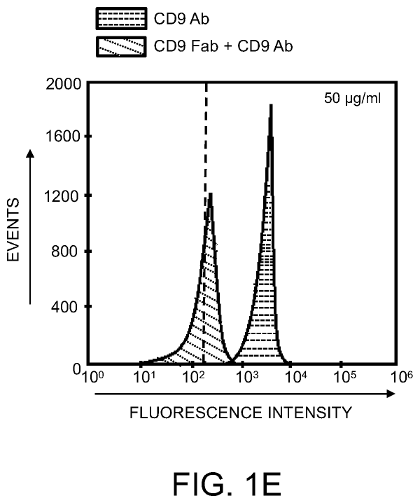

In another embodiment, for analysis purposes, the cells are trypsinized (0.05% trypsin with 0.53 nM EDTA), washed with a blocking buffer, comprising 10% bovine serum albumin (BSA), and placed on ice. Subsequently, the cells are incubated with CD9 Fab fragment or without, anti-CD9 mAb at various concentrations, and fluorescein isothiocyanate (FITC)-conjugated Fc-specific anti-mouse secondary antibody, for 30 min each in the cold. The cells are washed twice with PBS after each antibody staining. The cells are analysed using an SH800 cell sorter with a PMT voltage of 40% and a flow rate in a range of 100 events to 200 events per second. At least 40,000 total events, for example, were collected when implementing embodiments of the present disclosure (FIGS. 1D, 1E).

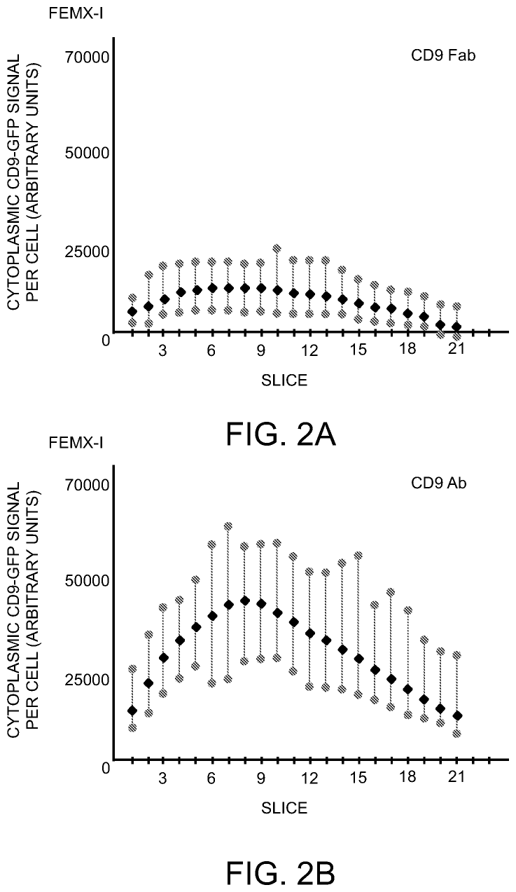

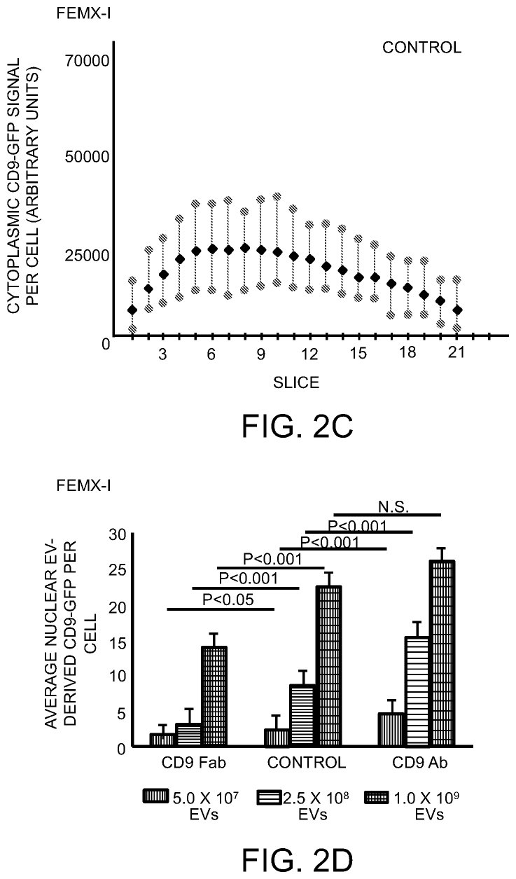

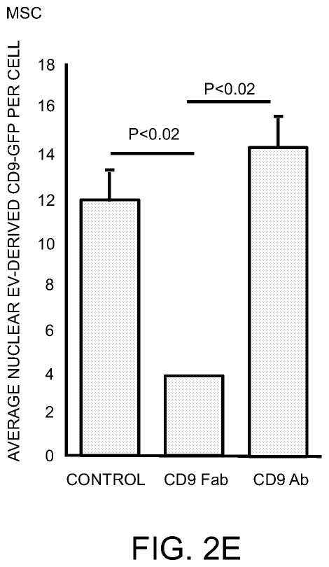

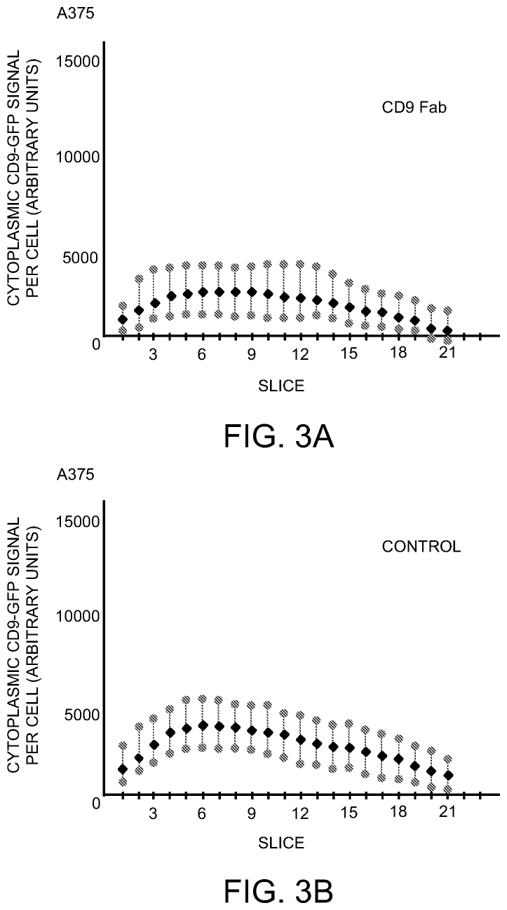

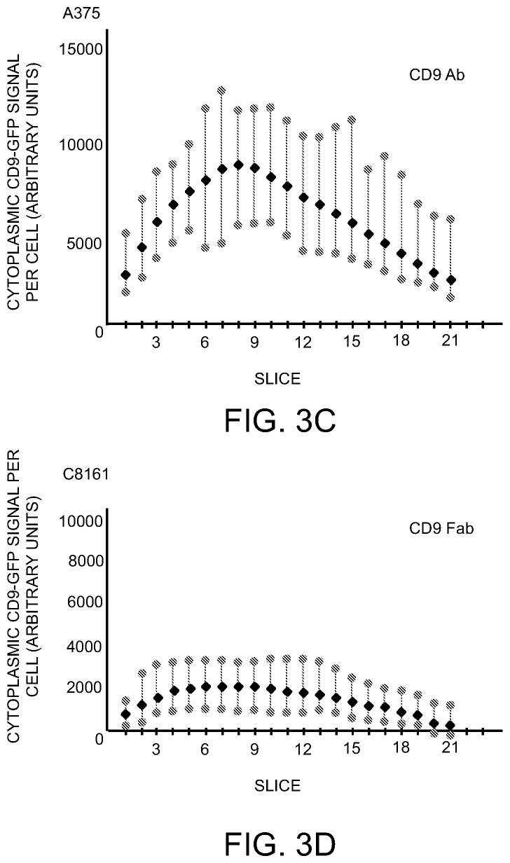

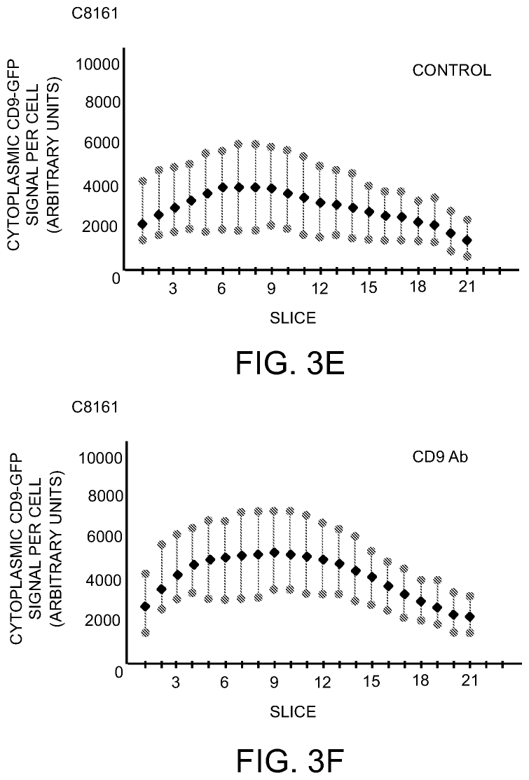

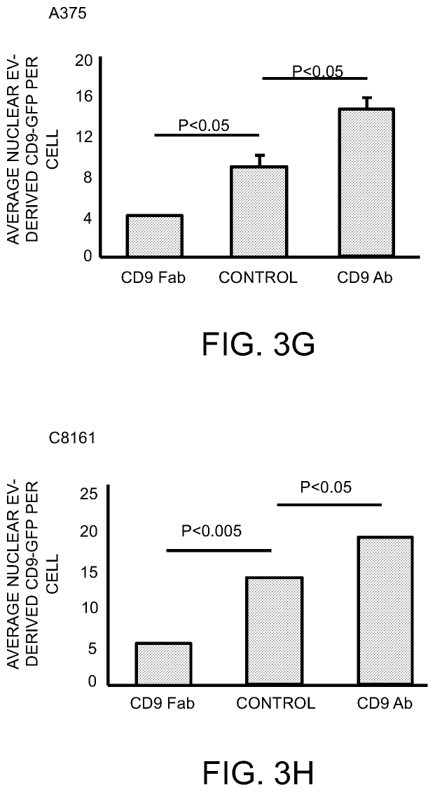

To determine the impact of CD9 Fab on the endocytosis of EVs by melanoma cells, engineered cells, for example, FEMX-I cells, are beneficially used to express the CD9-GFP fusion protein. These cells release in vivo-labeled fluorescent EVs that can be used to monitor EV uptake upon incubation with recipient cells. CD9-GFP⁺ EVs released in the conditioned culture media are beneficially enriched by differential centrifugation. Prior to the exposure of native FEMX-I cells to CD9-GFP⁺ EVs, the cells are pre-incubated for 30 minutes at a temperature of 37° C. with either CD9 Fab or CD9 Ab (25 μg/ml). As control, no antibody is added. Afterward, cells are cultured with CD9-GFP⁺ EVs (2.5×10⁸ particle/ml) without removing the antibodies for 5 hours and then fixed, immunolabeled for protein SUN domain-containing protein 2 (SUN2), an inner nuclear membrane protein, and analysed by CLSM. A three-dimensional (3D) reconstruction of labeled recipient cell suggests that CD9-GFP signal associated with their cytoplasm is considerably reduced in the presence of CD9 Fab as compared to a control. In contrast, the presence of CD9 Ab yielded the opposite effect, namely an increase of cytoplasmic CD9-GFP is detected (FIGS. 2A, 2B, 2C). Additionally, similar outcomes are observed with two other melanoma cell lines, A375 and C8161 exposed to FEMX-I cell-derived CD9-GFP+ EVs, indicating that CD9 Fab inhibits the uptake of EVs (FIGS. 3A-F).

It will be appreciated that the EV-derived cargo proteins are not only endocytosed by the host cells, but also a fraction of endocytosed EV-derived cargo proteins is transferred to their nucleoplasm by the intermediate of late endosomes entering into the nucleoplasmic reticulum. The analysis of the nuclear compartment of melanoma cells pre-treated with monovalent or divalent antibodies prior to incubation with CD9-GFP+ EVs (2.5×10⁸ particle/ml) shows an increase or decrease in CD9-GFP+ signals in the nucleoplasm, respectively, compared to the control. Furthermore, the addition of different amounts of CD9-GFP+ EVs (for example, 5.0×10⁷ or 1.0×10⁹ particle/ml) in FEMX-I cells shows that the numbers of nuclear CD9-GFP are significantly lower or higher in cells exposed to CD9 Fab or CD9 Ab, respectively. Moreover, only with a high amount of EVs (i.e. 1.0×10⁹ particle/ml) no significant difference is observed between CD9 Ab and control (FIG. 2D). Furthermore, similar observations are made with A375 and C8161 cells (FIGS. 3G, 3H).

Furthermore, to determine the impact of CD9 antigen-binding fragment (CD9 Fab) on the infection of HIV virus into recipient cells, HeLa cells are beneficially used to express the HIV virus protein. HeLa cells are pre-incubated for 30 min with CD9 Fab monovalent (50 μg/ml) prior to HIV infection. Additionally, the HIV virus is pre-incubated at 4° C. prior to addition to HeLa cells. Alternatively, both the HeLa cells and HIV virus are pre-incubated with the CD9 Fab monovalent, and subsequently the HIV virus is added to the HeLa cells. The analysis of the HeLa cells expressing HIV virus protein pre-incubated with CD9 Fab monovalent shows an increase inhibition of the HIV viral infection in the HeLa cells as compared to the individual inhibition of HIV virus outside the HeLa cells. Hence, the CD9 Fab monovalent is effective in blocking the infection from the HIV virus.

### DESCRIPTION OF DRAWINGS

Referring to FIGS. 1A and 1B, there are depicted pictorial representations of generation of a CD9 antigen-binding fragment (CD9 Fab). Notably, intercellular communication is mediated by extracellular membrane vesicles (EVs). EVs are endocytosed (taken up) by a recipient (host) cell and endocytosed EV-associated proteins are transported into nucleoplasm of the recipient cell through its nucleoplasmic reticulum. Furthermore, a presence of a tetraspanin CD9 on the EVs mediate the endocytosis of EVs and the nuclear transfer of the endocytosed EV-associated proteins.

FIG. 1A depicts a 5H9 anti-CD9 monoclonal antibody or CD9 antigen-binding fragment (CD9 Fab) being targeted at its antigen, the tetraspanin CD9. It will be appreciated that targeting the tetraspanin CD9 in the EVs (and optionally, the recipient cell) substantially decreases the endocytosis of EVs and abolishes the nuclear transfer of the endocytosed EV-associated proteins.

FIG. 1B depicts a stepwise protocol for the generation of a CD9 antigen-binding fragment (CD9 Fab) via a papain digestion of a full-size 5H9 anti-CD9 monoclonal antibody. The full-size 5H9 anti-CD9 monoclonal antibody referred to as IgG undergoes papain digestion to result in a digested IgG comprising a CD9 fragment crystalline (CD9 Fc) component and a pair of CD9 antigen-binding fragment (CD9 Fab) components. It will be appreciated that the derived CD9 Fc is extracted with Protein A, while CD9 Fab is recovered in the flow through.

Referring to FIG. 1C, there is provided a schematic illustration depicting full-size 5H9 anti-CD9 monoclonal antibody and Fab fragments thereof upon papain digestion. Typically, the full-size 5H9 anti-CD9 monoclonal antibody (Ab) is incubated with papain-immobilized agarose resin for digesting the full-size 5H9 anti-CD9 monoclonal antibody. After digestion, the CD9 antigen-binding fragment (CD9 Fab) and the CD9 fragment crystalline (CD9 Fc) components are separated by Protein A in a depletion treatment of the papain-immobilized agarose resin. The full-size 5H9 anti-CD9 monoclonal antibody indicated by Ab, the CD9 antigen-binding fragment (CD9 Fab) indicated by Fab, and the digested and reduced CD9 antigen-binding fragment (CD9 Fab) indicated by asterisk (*), are assessed by SDS-PAGE gel electrophoresis and stained with Coomassie brilliant blue. Notably, the SDS-PAGE gel electrophoresis result shows the full-size 5H9 anti-CD9 monoclonal antibody (Ab) before papain digestion (left lines) and CD9 antigen-binding fragment (CD9 Fab) components after papain digestion and reduction with Protein A (right lines).

Referring to FIGS. 1D and 1E, there is illustrated a graphical representation of data depicting the binding of 5H9 anti-CD9 monoclonal antibody (CD9 Ab) to melanoma cells in presence and absence of CD9 antigen-binding fragment (CD9 Fab) at an antibody concentration of 25 μg/ml (1D) and 50 μg/ml (1E). Notably, the binding of 5H9 anti-CD9 monoclonal antibody (CD9 Ab) to melanoma cells is inhibited by the CD9 antigen-binding fragment (CD9 Fab). Typically, melanoma cells, such as FEMX-I cells, are sequentially surface labelled with the CD9 antigen-binding fragment (CD9 Fab) followed by the 5H9 anti-CD9 monoclonal antibody (CD9 Ab) and a fluorochrome (such as fluorescein isothiocyanate or FITC) conjugated Fc-specific secondary antibody. A flow cytometry analysis of the FEMX-I cells labelled with the 5H9 anti-CD9 monoclonal antibody (CD9 Ab) and the fluorochrome conjugated Fc-specific secondary antibody results in a standard curve. The standard curve, generated using the median channel values, indicates the amount of CD9s per cell. The pre-incubation of cells with CD9 antigen-binding fragment (CD9 Fab) prior the 5H9 anti-CD9 monoclonal antibody (CD9 Ab) and the fluorochrome-conjugated Fc-specific secondary antibody incubations, reduces the binding of CD9 Ab to melanoma cells at 25 μg/ml concentration of the antibodies (1D) and 50 μg/ml concentration (1E). In other words, targeting the CD9 Fab in recipient cell (and optionally in EVs) with higher concentrations of CD9 Fab substantially decreases the amount of available CD9s per cell.

Referring to FIGS. 2A, 2B and 2C, there are illustrated graphical representations of data depicting a significance of CD9 antigen-binding fragment (CD9 Fab) in the uptake of extracellular membrane vesicles (EVs) by melanoma (FEMX-I) cells. As aforementioned, endocytosis of EVs by a recipient cell occurs through their penetration into cell membranes and through cytoplasm of the recipient cell. Melanoma cells, such as FEMX-I cells, are pre-incubated with CD9 antigen-binding fragment (CD9 Fab) (as shown in FIG. 2A) or anti-CD9 monoclonal antibody (CD9 Ab) (as shown in FIG. 2B) or without either of the CD9 Fab and CD9 Ab (Control) (as shown in FIG. 2C) and later exposed to fluorescent EVs derived CD9-GFP⁺ FEMX-I cells. The samples are further immunolabeled for protein SUN domain-containing protein 2 (SUN2) prior to analysis by confocal laser scanning microscopy (CLSM). It will be appreciated that SUN2 is an inner nuclear membrane (INM) protein associated with nuclear envelope. The amounts of cytoplasmic CD9-GFP signals per cell are quantified over 0.4 μm slices from 10 individual cells.

FIGS. 2D and 2E are graphical representations of data depicting a significance of CD9 antigen-binding fragment (CD9 Fab) in the nuclear transfer of EV-derived CD9-GFP by melanoma (FEMX-I) cells (D) and mesenchymal stem cells (MSC) (E) respectively. Melanoma cells, such as FEMX-I cells are pre-incubated with CD9 Fab or CD9 Ab or without either of the CD9 Fab and CD9 Ab (Control) and later exposed to fluorescent EVs derived CD9-GFP⁺ FEMX-I cells. Different concentrations of EVs, such as 5.0×10⁷ particle/ml, 2.5×10⁸ particle/ml, or 1.0×10⁹ particle/ml, are used for all the three aforementioned cases, i.e. FEMX-I cells pre-incubated with CD9 Fab or CD9 Ab and as the Control. The samples are further immunolabeled for SUN2 prior to analysis by confocal laser scanning microscopy (CLSM). The amounts of nuclear EV-derived CD9-GFP signals per cell are quantified over 0.4 μm slices from 10 individual cells (as shown in FIG. 2D).

In FIG. 2E, the mesenchymal stem cells (MSC) are exposed to EVs, at a concentration of 1.0×10⁹ particle/ml, derived CD9-GFP⁺ MSCs upon a pre-incubation with CD9 Fab or CD9 Ab or without either of the CD9 Fab and CD9 Fab (Control). The samples are further immunolabeled for SUN2 prior to analysis by confocal laser scanning microscopy (CLSM). The amounts of nuclear EV-derived CD9-GFP signals per cell are quantified over 0.4 μm slices from 10 individual cells.

Referring to FIGS. 3A to 3C and 3D to 3F, there are illustrated graphical representations of data depicting significance of CD9 antigen-binding fragment (CD9 Fab) in the uptake of EVs by melanoma cells, A375 and C8161. Notably, CD9 Fab impedes the uptake of EVs in various malignant melanoma cells, such as A375 (as shown in FIGS. 3A-3C) and C8161 (as shown in FIGS. 3D to 3F). A375 and C8161 cells are incubated without (CONTROL) (as shown in FIG. 3B and FIG. 3E respectively) or with CD9 Fab (as shown in FIG. 3A and FIG. 3D respectively) or 25 μg/ml concentration of the CD9 Fab (as shown in FIG. 3C and FIG. 3F respectively). The samples are further exposed to fluorescent EVs, at a concentration of as 2.5×10⁸ particle/ml, wherein the fluorescent EVs are derived from FEMX-I cells expressing CD9-GFP. Furthermore, the samples are immunolabeled for SUN2 and analysed using confocal laser scanning microscopy (CLSM). The amounts of cytoplasmic CD9-GFP signals per cell are quantified over 0.4 μm slices from 10 individual cells (A375 and C8161).

Referring to FIGS. 3G and 3H, there are illustrated graphical representations of data depicting significance of CD9 antigen-binding fragment (CD9 Fab) in the nuclear transfer of EV-derived CD9-GFP by melanoma cells, A375 and C8161. Notably, CD9 Fab impedes the uptake of EVs and nuclear transfer of their cargo proteins in various malignant melanoma cells, such as A375 (as shown in FIG. 3G) and C8161 (as shown in FIG. 3H). A375 and C8161 cells are incubated without (CONTROL) or with CD9 Fab or CD9 Ab at 25 μg/ml concentration. The samples are further exposed to fluorescent EVs, at a concentration of as 2.5×10⁸ particle/ml, wherein the fluorescent EVs are derived from FEMX-I cells expressing CD9-GFP. Furthermore, the samples are immunolabeled for SUN2 and analysed using confocal laser scanning microscopy (CLSM). The amounts of average nuclear EV-derived CD9-GFP signals per cell are quantified over 0.4 μm slices from 10 individual cells (A375 and C8161).

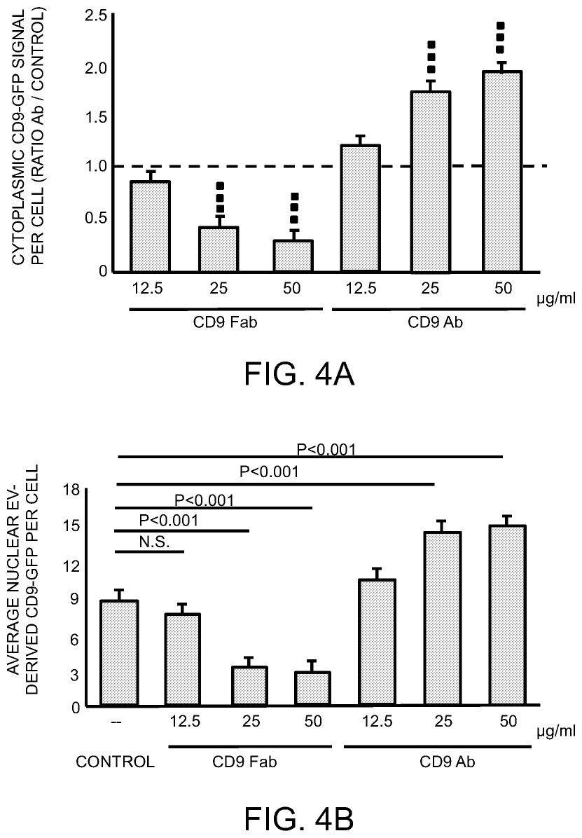

Referring to FIGS. 4A and 4B, there are illustrated graphical representations of data depicting dose-dependent inhibition of CD9 antigen-binding fragment (CD9 Fab) on the uptake of EVs and nuclear transfer of EV-derived CD9-GFP by melanoma (FEMX-I) cells. The FEMX-I cells are pre-incubated with different concentrations of CD9 Fab or CD9 Ab as indicated, prior to the exposure to CD9-GFP⁺ EVs, at a concentration of 2.5×10⁸ particle/ml. It will be appreciated that no antibody was added in CONTROL, and the relative fluorescence of CD9-GFP in the cytoplasm of recipient cells is evaluated by comparison to the CONTROL from 10 individual cells (as shown in FIG. 4A). The nuclear CD9-GFP signals per cell are quantified over 30 individual cells (as shown in FIG. 4B).

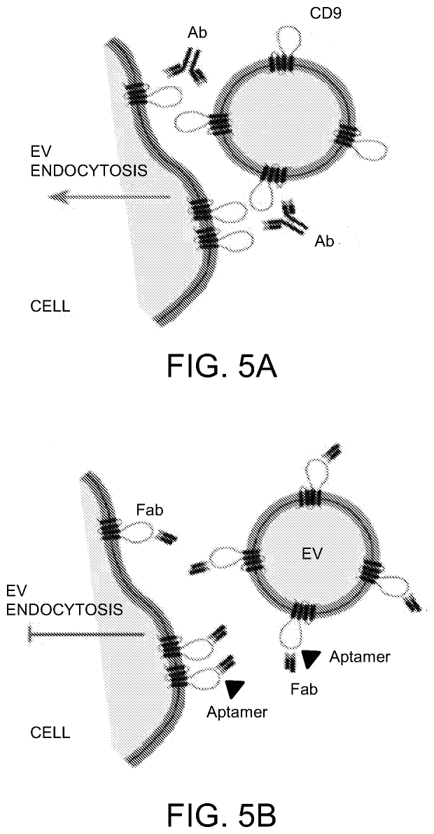

Referring to FIGS. 5A and 5B, there are depicted pictorial representations displaying a positive and negative impact of 5H9 anti-CD9 monoclonal antibody (CD9 Ab) and CD9 antigen-binding fragment (CD9 Fab) and/or aptamer, respectively, on the endocytosis of CD9-containing EVs. CD9 mediates the cytoplasmic uptake of EVs and nuclear transfer of EV-associated proteins in the recipient cell (as shown in FIG. 5A). The CD9 Fab and/or the aptamer impedes the uptake of EVs and nuclear transfer of their cargo proteins in various malignant melanoma cells and mesenchymal stem cells (MSC) by binding with the CD9 antigen expressed on the EVs and/or the recipient cells (as shown in FIG. 5B).

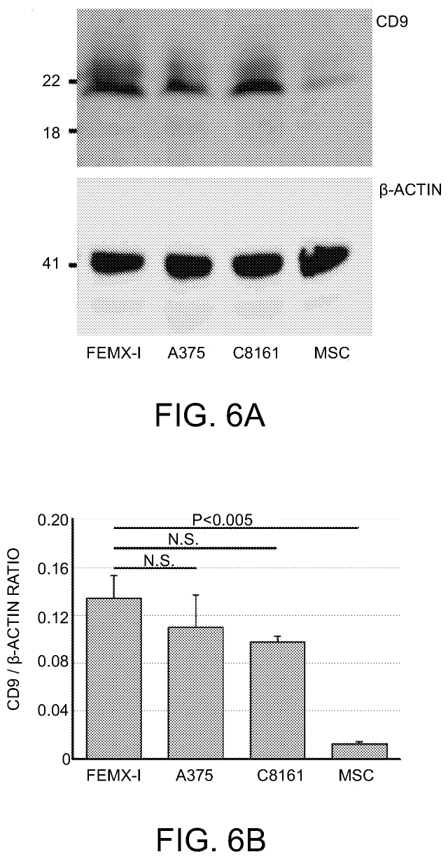

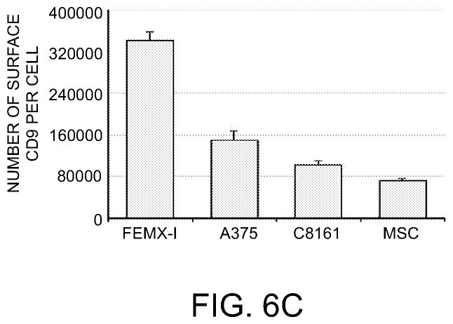

Referring to FIG. 6A, shown is pictorial representation depicting a quantification of CD9 and β-actin in melanoma cell lines (FEMX-I, A375 and C8161) and primary mesenchymal stem cells (MSC). Detergent lysates prepared from melanoma cell lines (FEMX-I, A375 and C8161) and primary mesenchymal stem cells (MSC) are probed for CD9 and β-actin using immunoblotting technique.

Referring next to FIG. 6B, there is illustrated a graphical representation of data depicting quantification of CD9 and β-actin in melanoma (FEMX-I, A375 and C8161) cells and primary mesenchymal stem cells (MSC). A ratio of CD9 to β-actin, i.e. CD9/β-actin, immunoreactivities are shown in melanoma (FEMX-I, A375 and C8161) cells and primary mesenchymal stem cells (MSC).

Referring to FIG. 6C, there is illustrated a graphical representation of data depicting a number of surface CD9 per cell in melanoma cell lines (FEMX-I, A375 and C8161) and primary mesenchymal stem cells (MSC).

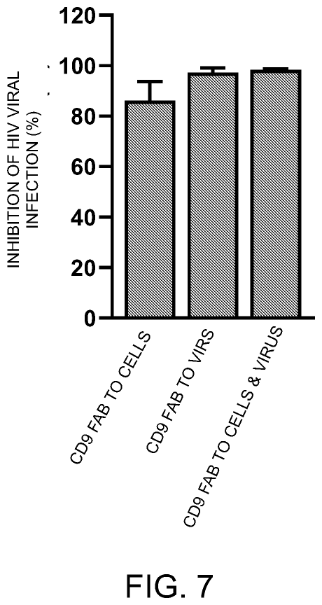

Referring to FIG. 7, there is illustrated a graphical representation of data displaying the impact of CD9 antigen-binding fragment (CD9 Fab) on the infection of HIV virus into recipient cells. As shown, the CD9 Fab monovalent inhibits the HIV viral infection in HeLa cells (control), in HIV virus as well as in HeLa cells expressing HIV virus protein, wherein the inhibition of the HIV viral infection in the HeLa cells expressing HIV virus protein is highest as compared to the individual inhibition of HIV virus and the HeLa cells. Hence, the CD9 Fab monovalent is effective in blocking the infection from the HIV virus.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. An antigen-binding fragment (Fab) and/or an aptamer that recognises an target epitope in an extracellular part of CD9, wherein the Fab and/or the aptamer bind directly to the extracellular part of the CD9, and wherein the Fab binds to the extracellular part of the CD9 at a similar location as that of the aptamer.

2. An antigen-binding fragment (Fab) according to claim 1, wherein the Fab is a fragment of a monoclonal antibody and has a size of approximately 40-60 kDa, 45-55 kDa, 48-52 kDa or 50 kDa, and wherein the monoclonal antibody is at least one of an anti-human CD9 and an anti-CD9.

3. An antigen-binding fragment (Fab) according to claim 1 or 2, wherein the monoclonal antibody is an antibody derived from at least one of antigens including TSPAN29, DRAP-27, BTCC-1, motility related protein-1, leukocyte antigen MIC3, 5H9, MRP-1 and BA-2/P24.

4. An antigen-binding fragment (Fab) according to any of claims 1 to 3, wherein the fragment of the monoclonal antibody (CD9 Fab) is derived from a 5H9 anti-CD9 monoclonal antibody, optionally wherein the fragment is derived by digestion of the antibody with papain.

5. An antigen-binding fragment (Fab) according to claim 4, wherein the 5H9 anti-CD9 monoclonal antibody fragment binds directly to the extracellular part of the CD9.

6. An aptamer according to claim 1, wherein the aptamer is made of DNA or RNA and the aptamer is a CD9 aptamer.

7. An antigen-binding fragment (Fab) and/or an aptamer according to any of claims 1 to 6, wherein the antigen-binding fragment (Fab) and/or the aptamer inhibits an intercellular communication in multicellular organisms by interfering with at least one of:
(i) an endocytosis of extracellular membrane vesicles (EVs);
(ii) a fusion of EVs with cells; and
(iii) an intracellular transport of endocytosed biomaterials in recipient cells.

8. An antigen-binding fragment (Fab) and/or an aptamer according to any of claims 1 to 6 for use in at least one of medicine, veterinary medicine, agriculture or as an insecticide.

9. An antigen-binding fragment (Fab) and/or an aptamer according to any of claims 1 to 6 for use as a medicament.

10. An antigen-binding fragment (Fab) and/or an aptamer according to any of claims 1 to 6 for use in the treatment and/or prevention of a disease or condition in which the endocytosis of extracellular vesicles is implicated, optionally wherein the subject is a human.

11. An antigen-binding fragment (Fab) and/or an aptamer for use according to claim 9 or 10, wherein the disease or condition includes at least one of cancer, cancer metastasis and an infection.

12. An antigen-binding fragment (Fab) and/or an aptamer for use according to claim 11, wherein the infection is caused by a virus.

13. An antigen-binding fragment (Fab) and/or an aptamer for use according to claim 12, wherein the virus is an enveloped virus, optionally the virus is HIV-1.

14. An antigen-binding fragment (Fab) and/or an aptamer for use according to claim 9 or 10, wherein the disease is a neurodegenerative disease.

15. An antigen-binding fragment (Fab) and/or an aptamer for use according to claim 9 or 10, wherein the disease is Alzheimer's disease, Parkinson's disease, ventricular hypertrophy, a type I diabetes, a type II disease, a kidney disease, macular degeneration or a lung disease.

16. A pharmaceutical composition comprising an antigen-binding fragment (Fab) and/or an aptamer as defined in any preceding claim, and at least one pharmaceutically acceptable excipient, adjuvant or carrier.

17. A pharmaceutical composition according to claim 16 for use in the treatment and/or prevention of a disease or condition selected from cancer, cancer metastasis, infection, a neurodegenerative disease, Alzheimer's disease, Parkinson's disease, ventricular hypertrophy, a type I diabetes, a type II disease, a kidney disease, macular degeneration and a lung disease.

18. An antigen-binding fragment (Fab), an aptamer or a pharmaceutical composition for use according to any preceding claim wherein the patient is further administered one or more therapeutic agents or when the Fab and/or the aptamer is provided in combination with one or more therapeutic agents.

19. A pharmaceutical composition according to claim 18 for use in combination with one or more therapeutic agents for the treatment and/or prevention of the disease or condition.

20. A biological molecular assay that recognises a target epitope in an extracellular part of CD9, wherein the biological molecular assay includes the use of an antigen-binding fragment (Fab) and/or an aptamer, and wherein the Fab and the aptamer bind directly to the extracellular part of the CD9, and wherein the Fab binds to the extracellular part of the CD9 at a similar location as that of the aptamer.

21. A biological molecular assay of claim 20, wherein the Fab is a fragment of a monoclonal antibody and has a size of approximately 40-60 kDa, 45-55 kDa, 48-52 kDa or 50 kDa, and wherein the monoclonal antibody is at least one of an anti-human CD9 and an anti-CD9.
